(19) United States
(12) Patent Application Publication
(10) Pub. No.: US 2023/0285490 A1
(43) Pub. Date: Sep. 14, 2023

(54) ADDITIVE TO FISH FOOD COMPRISING TWO PLANT EXTRACTS AND A SEAWEED (PALMARIA PALMATA) EXTRACT AS TASTE MASKING AGENT; AND A METHOD TO COMBAT/ELIMINATE INFESTATIONS OF CALIGUS BY STERILIZATION OF FEMALE INDIVIDUALS IN FISH FARMING CENTERS AND MARINE CONCESSIONS

(71) Applicant: ACUANATIVA SPA, Maule (CL)

(72) Inventor: Marcelo Julio VELASCO REYES, Recoleta Santiago (CL)

(21) Appl. No.: 18/040,758

(22) PCT Filed: Aug. 5, 2021
(86) PCT No.: PCT/CL2021/050075

§ 371 Date: Feb. 6, 2023

(30) Foreign Application Priority Data
Aug. 6, 2020 (CL) 2045-2020

(51) Int. Cl. A61K 36/58; A61K 36/67; A61K 47/46; A61P 33/14; A23K 10/30; A23K 50/80

(52) U.S. Cl. CPC A61K 36/58; A61K 36/67; A61K 47/46; A61P 33/14; A23K 10/30; A23K 50/80

(57) ABSTRACT

The present invention is related to aquiculture, particularly, to an additive to fish food comprising two plant extract and a seawater extract selected from Palmaria palmata as taste masking agent and method for combating ectoparasites in fishes. Even more, particularly, a food additive and method for combating/eliminating Caligus in fish farming centers and ocean concessions resulting in the sterilization of ectoparasite female.

### FIELD OF THE INVENTION

The present invention is related to aquiculture, particularly, to a fish food additive comprising two plant extracts and a seaweed extract selected from Palmaria palmata as a taste masking agent, and method for combating fish ectoparasites. Even more, particularly, to a food additive and method for combating/eliminating Caligus in fish farming centers and marine concessions, resulting in the sterilization of female ectoparasites.

### PRIOR ART

Oftenly ectoparasite infestations occurring in fish farming centers, living in the fish skin or skin excretions (hosts) during long periods of time, causing a severe damage in fishes by chronic stress and oportunitic pathogen infections. Mostly these ectoparasites are specific hosts, and in salmon and trout cultures, the main and the most serious infestation problem corresponds to crustaceous ectoparasites. Among them, the most predominant is copepode arthropode, Caligus rogercresseyi(sea slice), being a serious problem since the same is feeding of host blood, mucus and epidermic tissue. Thus, they can fixed to the sekin of fishes commonly causing haemorrages and spongy and necrotic wounds at the fixing sites.

Generally, sea slices having a simple life cycle comprising the following stages/stages: junior adjuvants (parasites) and mobile pre-adults (free swimming) and adult stages in the host. Pregnant female individuals produce a serie of egg chains, to give raise to three planctonic free life stages before the settlement in a host. Exact stages of a life cycle depend on the specie. No parasitic sea slice stage corresponds to those having free swimming while the parasitic stage corresponds to those in which the parasite require feeding. Ectoparasites have further a certain time to find a host, and thus, the distance to which this host is located plays an important role in the infestations. Eggs hatching from pregnant female individuals, transforming into free swimmig larvae along the time. An average time range between parasite hatching and its infectious capacity is about of 4 days at 10° C. and 2 days at 15° C. Larvae can freely swim through superficial ocean currents, and after found a host, they can fix/infect the same, starting a feeding stage.

After feeding, sea slices can be specially fixed at the frontal (head), dorsal (back) and perineal regions of fishes, causing serious damages in the skin than, in absence of a treatment can cause fish dead by severe erosion and subcutaneous tissue exposition, secondary bacterial infections, osmotic imbalance, extreme stress, among other causes. It should be remarked that, parasitic effect generally depends on the fish species, aging and general sanitary condition. Additionally, the managing of sea slices must consider biological factors as ectoparasite life-cycle step as well as environmental conditions, including ocean current and water temperature.

The aquiculture industry combines a preventive practice and approved/authorized treatments, which allow combating ectoparasite infestations under certain life-cycle steps and a certain water temperature. Generally, these treatments are administered by food or topical baths, and mostly of them are not environmentally innocuous or friendly. An alternative which reduces environmental effects of fish parasiticides, although limited in validation, acceptance, and extension of application, is the ectoparasite control by depredation of ocean species which are natural predators. Under any of the above-mentioned strategies, special qualified persons are required to supply treatment or specific implements around fish farming cages, without obtaining a high effectivity percent in prevention or treatment of infestation or re-infestation, despite the intensive labor of the procedure. This type of treatment is very stressful to the fish and frequently it is not used.

Nevertheless, despite the preventive measurements and existent treatments, the ocean ectoparasite infestation continue being an important problem to the aquiculture industry since added to a low efficiency of treatments, sea slices have developed a resistance against the mentioned treatment, and thus, there is a growing necessity of counting with alternative methods Improving the antiparasitic treatments with an ecosystemic and environmental look according to a clean production in aquiculture.

In relation to scientific publications or patents, it is possible to particularly mention: WO2015021534A1 (Sylleta Inc) providing a limonoid composition to the prevention and control of ocean ectoparasites in fishes, wherein such limonoid is selected from the group consisting of azadirachtin, salanine, meliantriol, karanjin and nimbin or is a Neem, Milletia pinnata or Azadirachta indica extract. This composition can be incorporated to the food by microencapsulation, using a determined polymer (forming a type of net or matrix wherein the compound is found) or is directly incorporated in the fish food.

WO2014140623A1 (Neemco Ltd) is related to a food to fishes to preventing, treating and/or controlling a variety of diseases, infections and/or infestations in fishes, comprising limonene, azadirachtin; compositions, uses and methods to prepare supplemented fish food. Song et al. (2017); Transcriptomics and proteomic studies reveal acaricide mechanism of octadecanoic acid-3,4-tetrahydrofuran diester against Sarcoptes scabiei var. Cuniculi. Sci. Rep. 7, 45479; doi:10.1038/srep45479, is related to octadecanoic-3,4-tetrahidrofurane acid diester, having a potent acaricide activity from neem oil, and proposing the acaricide mechanism of such compound against Sarcoptes scabiei var. cuniculi (mite associated to scabies), remarking that after a treatment with octadecanoic-3 acid, 4-tetrahidrofurae diester, mite genes related to energetic metabolism are significantly regulated up/downward, included cytrate cycle, the oxidative phospholiration pathway and fatty acid metabolism. Although divergent regulating effects are showed by octadecanoic-3-tetrahidrofurane acid or 4-tetrahidrofurane diester.

Jones et al., (2006); Reduced sensitivity of the salmon louse, Lepeophtheirus salmonis, to the organophosphate dichiorvos. J. Fish Dis. 15, 197-202. 10.1111/j.1365-2761.1992.tb00654.x. https:llonlinelibrary.wiley.com/doi/abs/10.1111/i.1365-2761.1992.tb00654.x is related to the use of an organophosphrated plaguicide, dichiorvos (2,2-dichlorovinyl-dimethyl phosphate) in pisciculture to the reduction in the sensibility of salmon slices, particularly, Lepeophtheirus salmonis, describing that this slice showing differences in the sensibility to dichiorvos.

Lu, et al., (2018). Deltamethrin toxicity: A review of oxidative stress and metabolism. Environ Res. 170, 260-281. doi: 10.1016fj.envres.2018.12.045. httos://www.sciencedirect.com/science/article/abs/oii/SO013935118306819?via %3Dihub is related to deltamethrin, a pyrethroid having a valuable insecticide activity against plagues and parasites, its mechanism of toxicity from a perspective of direct or mediated oxidative stress perspective, oxidative signal pathways and modulating effects of antagonists, synergists and placebos oxidative stress and reviewing its metabolism, including metabolites, metabolic enzymes and toxicity pathways.

Thus, the necessity of counting with effective and friendly environmental treatments to combating/eliminating ectoparasites from fish farming centers and ocean concessions, persists.

The present invention proposes a fish food additive and method for combating fish ectoparasites.

Specifically, the present invention proposes a fish food additive and method for combating/eliminating Caligus in fish farming centers and ocean concessions, associated to an method for sterilizing ectoparasite. This fish food additive comprising a mixture of 2 plant extracts and a seaweed extract selected from Palmaria palmata as taste masking agent.

### BRIEF DESCRIPTION OF DRAWINGS

FIG. 1. Showing an average parasite loading per diet, 9 days post-infestation.

FIG. 2. Showing an average parasite loading per diet, 20 days post-infestation.

FIG. 3. Showing a Caligus percent as development stage per tank and diet.

FIG. 4. Showing a number composition of mobile Caligus adults per tank and diet.

FIG. 5. Showing a Caligus percent of mobile adults per tank and diet.

FIG. 6. showing the effectivity in the development control of Caligus rogercresseyi embryos inside ovigerous sacs under controlled laboratory conditions (in vitro assay).

### DETAILED DESCRIPTION OF THE INVENTION

The present invention is related to an additive to fish food comprising: (i) a mixture of a first plant extract selected from a nimbus or Indian margosa extract, (ii) a second plant extract selected from pepper, and (iii) a seaweed extract selected from Palmaria palmata as taste masking agent. This additive allows the control of Caligus infestation, caligidosis, limiting the larvae development and sterilizing female parasite adult stages of fishes fed with the same.

Fish food additive can be incorporated in food at dose ranging 200-4000 grams per food ton, preferably ranging 500-2000 grams per food ton, and even more preferably 200-500 grams per food ton.

Fish food additive of the present invention, useful in the Caligidosis prevention can be orally or topically administered: 1) as powder premix to fish food; 2) as powder, in oil, prior to oiling the food; 3) in capsules together with one or more fish antiparasitic treatments; 4) in gel solutions to feeding fishes; or 5) combining with additional palates to be incorporated in fish food.

Fish food additive is prepared from mixing a first plant extract selected from nimbus extract or indian margosa extract and a second plant extract selected from pepper and a seaweed extract selected from Palmaria palmata as taste masking agent. Weight ratio between first botanical extract: second botanical extract: seaweed taste masking agent ranging 70:10:20 to 50:5:45, preferably 65:5:30.

Fish food additive of the present invention is useful in the control of parasitosis limiting the larvae development and sterilizing parasite adult stages of fishes fed with the same. The present invention is also related to a method for combating/eliminating Caligus infestations in fish farming centers and ocean concessions, including sterilizing Caligus by supplying the food additive of the present invention.

Following, examples illustrating the invention without limiting the same:

### Example 1: Obtention of First Plant Extract

Azadirachta indica A. Juss fresh leaves were recollected and identified. Leaves were dried at air, comminuted, and pulverized in fine powder and stored in a glass vessel at 4° C. 250 g powder sample exhaustively extracted with distilled water using a reflux method (Trease GE, Evans WC. 1989. A textbook on pharmacognosy, 11th. edn. London, Bailliere Tinda). Mixture was filtered to remove remains. Subsequently, 200 ml petroleum ether was added to the mixture, strongly stirring and let settling. Petroleum ether layaer (upper part) was removed and concentrated. Subsequently, 200 ml chloroform was added to an aqueous layer and also strongly stirred and let settling. Crude aqueus layer is concentrated then up to an brown color extact was obtained using soft heating temperature. After weighed, labelled and sealed in a plastic vessel, and stored at −20° C. up to its use is required.

### Example 2: Obtention of Second Plant Extract

15 g milled large pepper are located into a Soxhelt apparatus, 250 ml, 150 ml ethanol 95% was added and 5 boiling chips and heated at reflux during 2 h. Mixture was filtered by suction filtration and then the filtrate was concentrated at a volume of 10-15 ml by simple distillation using a rotative evaporator. After weighed, labelled and sealed in a plastic vessel, and stored at 4° C. up to its use is required.

### Example 3: Obtention of Taste Masking Agent

Sweet water washing is performed to the seaweed Palmaria palmiata. Subsquently, the same is submitting to dry, remaining a wetting grade of 8% to submit to milling, up to obtain a powder of 400 microns.

### Example 4: Obtention of Fish Food Additive of the Present Invention

A direct combination/mixture of first and second plant extract with the seaweed taste masking agent was performed, in ratios p/p/p selected of 70:10:20 to 50:5:45.

### Example 5: Fish Administration/Feeding (In Vivo) with the Additive of the Present Invention to Caligus Control and Elimination

Table 1 stated the conditions in which fish bioassays were developed, if not expressly stated another condition:

TABLE 1
Parameters/conditions to bioassays

| Parameter | Acclimatization, challenge, medicated food relased |
|---|---|
| Water Temperature | 12.5° C. ± 1 |
| Photoperiod | 12 hours light/12 hours darkness |
| Water Replacement | 1.5 replacement/hour |
| Salinity | 31 ± 1 ppt |
| Oxygen | 80-120% saturation |
| Maximum Density | 20 kg/m³ |
| pH | 7.2-8.0 |

Table 2 showing the fish condition prior to assays/diet administration

TABLE 2
Average Weight Promedio 336 grams

| | | | | | | |
|---|---|---|---|---|---|---|
| Tank | 17 | 18 | 19 | 20 | 21 | 22 |
| Average Weigth (g) | 330 | 334 | 336 | 339 | 337 | 342 |
| Length (cm) | 30 | 30 | 30 | 30 | 31 | 31 |
| Estandar Desviation | 41 | 40 | 39 | 42 | 39 | 38 |
| CV (%) | 12 | 12 | 12 | 12 | 11 | 11 |
| K | 1.2 | 1.2 | 1.2 | 1.2 | 1.2 | 1.2 |
| Máx. | 400 | 406 | 406 | 400 | 400 | 402 |
| Mín. | 256 | 255 | 239 | 252 | 260 | 258 |
| Fishes/Tank | 50 | 50 | 50 | 50 | 50 | 50 |

### Assay Development

A total of 300 fishes of Atlantic Salmo (Salmo salar) were used having an average weight of 336 grams, which were distributed in 6 tanks, 1 m³, having 50 fishes each one, giving a starting to an acclimatation period of 12 days. Prior a conformation a sanitary check was made to the group to evaluate fish sanitary condition and rule out the pathogen presence. After the acclimationn period samples were taken prior to supplying diets (3 fish/tank) in ratios as stated in example 4. Further, Samples were taken to infestive copepodites in each tank prior to infestation. Subsequently, fishes of each tank were challenges with infestive Caligus rogercresseyi copepodites at a infestation pressure of 50 cop/fish (challenge protocol/infestation).

After the copepodite challenge (at 24 hours), a period of diet administration with the additive was started by triplicate using a ratio 70:10:20 (1st plant extract: 2nd plant extract: seaweed taste masking agent respectively, additive of the present invention). Diet Sea 1 (control) and diet Sea 2 (present invent) were administered: 1000 g/food ton (by triplicate) per 7 consecutive days. During this period, at post-infestation day 5, a fixation sampling was made to evaluate the sucess of the challenge at 5 fish/tank. Examples were returned post-counting to its respective tank. Followed the diet post-administration day samples were taken at 3 fish/tank which were eliminated. At post-infestation day 9, a counting was made to evaluate the parasitic load at the totality of fishes. With the counting results a second feeding period with the experimental diets was considered by 7 consecutive days. Ended this period a final sampling was made considering sampling weight/length, parasite counting and taking samples at 3 fish/tank.

As fish inclusion and exclusion criteria and after made an exploratory sampling to establish an average weight to the fish population destined to assay, only fishes showing a good adaptation condition and approved sanitary condition by a veterinary were included, excluding all the animal showing non appropriated conditions to bioassays. Diet and control were administered by triplicated. Codification of the received bags was as follows: Diet Sea 1 (2 bags; 24 kilograms app.) and Diet Sea 2 (2 bags; 26 kilograms app.). Both diets having caliber 4 mm. Diet Sea 2 contains the additive of the present invention as defined above in this example and the fish food ratio (Diet):additive above mentioned.

Fished were sampled in weight and length the 1st assay day and distributed in 6 tanks. Prior managing it, fishes were kept under fasting by 24 hours. Distribution was made aleatory to 10 fishes up to complete the required number. Average weight per group was 336 g. Prior to distribution, a sanitary check was performed in original tank to evaluate initial condition and discarding the pathogen presence (IPNv, SRS, BKD and Vibrio ordali).

Fishes were kept in acclimatation step per 12 days in sea water, in tanks, with a photoperiod of 12 hours light and 12 hours darkness, oxygen saturation between 80 and 120%, with an initial density of 7,4 Kg/m³.

2 relased periods of experimental diets were performed as stated the following feeding post-infestation protocol:
First period was performed 1 post-infestation day: where 2 diets were administered by triplicated by a period of 7 consecutive days.
Second period was performed 12 post-infestation days: where 2 diets were administered by triplicated by a period of 7 consecutive days.

After the acclimatation period, a challenge with Caligus rogercresseyi infestive copepodites were performed at an infestation pressure of 50 cop/fish (2.500 copepodites/tank). Infestations of each tank were made as stipulated in the above-mentioned challenge/infestation protocol. Infestations were made at retained water flow and total darkness by 6 hours, reducing the water level of each tank and supervising parameters each 30 minutes and supplying oxygen as required per time. Subsequently water flow and photoperiod were reset under normal operational conditions.

After infestation, 3 sampling of parasite loading were performed to observing and quantify stage and parasite number:
At post-challenge day 5, a fixation sampling was performed at 5 fish/tank. Individuals were sampled and returned to its original tank after sampled.
At post-challenge day 9, a parasite loading sampling was performed at the totality of fish/tank. Individuals were sampled and returned to its original tank after sampled.
At post-challenge day 20, a final sample was performed at the totality of fish/tank. Individuals were sampled o weight and length and eliminated at 24 hours post-counting.

During acclimatation fishes were fed at 1,12% SFR (Specific Fed Rate) as average. Food quantity per day was calculated and supplied in two rations per day: 70% an AM ration and 30% a PM ration. Non consumed food was recollected and weighed twice per day (12:30 h and 16:00 h), to estimate actual consumption/day. Commercial food, optimal Golden Biomar diet, was supplied (caliber 4 mm). During the administration of treatments, the determination of SFR gave the following results:

Period 1 (7 consecutive days), fishes of diet 1 consumed a rate of 1.32% SFR as average and fishes supplied with diet 2, 1.19% SFR as average.

Period 2 (7 consecutive days), fishes of diet 1 consumed a rate of 1.26% SFR as average and fishes supplied with diet 2, 1.19% SFR.

During the development of bioassays samples were taken at different time and steps. Sample taken were: Mucus, skin, muscle and liver, samples were subsequently stored (frozen at −20° C. or froozen at −80° C.).

An aleatory sampling was made of 5 fish/tank to evaluate the success of copopodite infestation (post-infestation day 5). It is remarked that this sampling was made after to a daily food routine with the purposes of not interrupting feeding with experimental diets. With respect to the Caligus life-cycle, it was observed that all the parasites adhered at fishes (to diet Sea 1 and diet Sea 2) corresponds to Chalimus I. A detail of average caligus N^{°}/tank is observed in Table 3.

TABLE 3
Fixation sampling per tank

| | | | | | | |
|---|---|---|---|---|---|---|
| Tank | 17 | 18 | 19 | 20 | 21 | 22 |
| Treatment | Sea 1 | Sea 2 | Sea 1 | Sea 2 | Sea 1 | Sea 2 |
| Average Caligus | 35.4 | 30.4 | 21.8 | 33.2 | 21.6 | 28.0 |
| Standard Desv. | 10 | 4 | 7 | 9 | 2 | 5 |
| CV | 27.9 | 12.4 | 32.3 | 26.1 | 8.4 | 19.6 |
| Máx. | 45 | 37 | 31 | 47 | 24 | 34 |
| Mín. | 22 | 28 | 12 | 24 | 19 | 20 |
| Parasite Total % Fixation | 177 | 152 | 109 | 166 | 108 | 140 |

It is observed that the fixation percent of all the tanks are all over 40% and parasite loading of all tanks are all over 20 caligus average. These values validate the success of the infestation and are within the established by protocol to an assay follows a course as planned. To fixation sampling an average load of 3 replica to diet Sea 1 was 26.3 caligus/fish and average parasite loading of 3 replicas to diet Sea 2 was 30.5 caligus/fish.

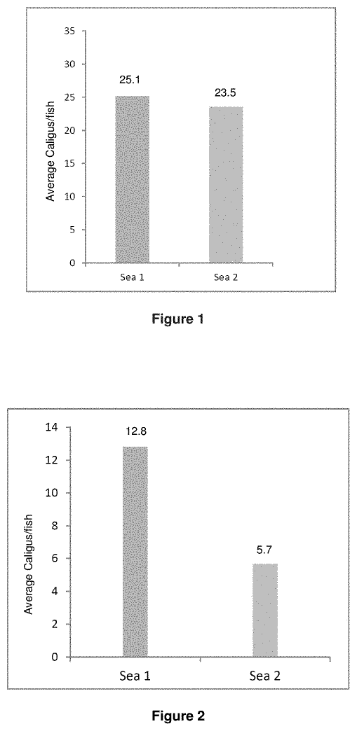

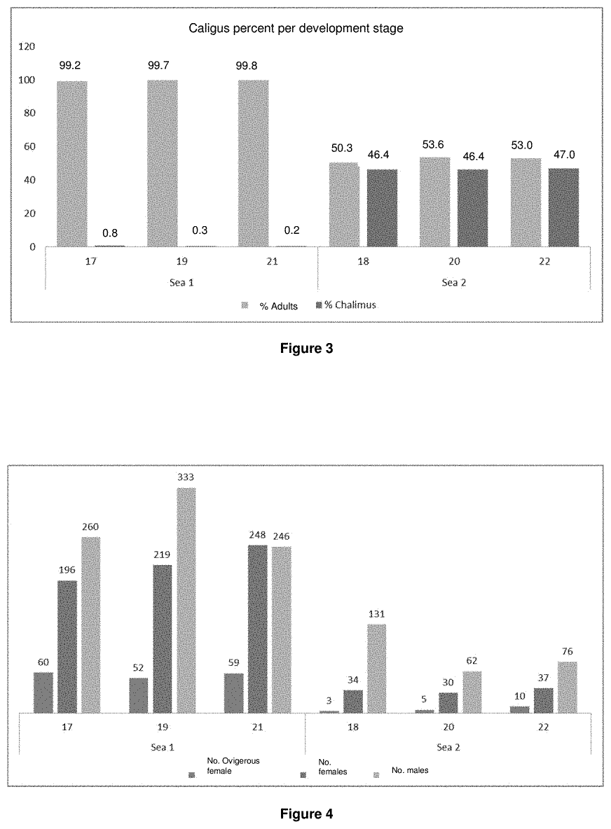

At 9 post-infestation days a sampling of parasite loading was made to the totality of fishes per tank (Table4). Average parasite loading of fishes in 6 analyzed tanks was 24.3 caligus/fish, where average loading per diet was 25.1 to Sea 1 and 23.5 to Sea 2 (FIG. 1). The highest average parasite loading was obtained in fishes of tank 17 (diet Sea 1) having 26 caligus/fish while the lowest average parasite loading was obtained to tank 20 (diet Sea 2) having 22.3 caligus/fish (Table 4). With respect to the caligus life cycle, it was observed that all the parasites adhered at fishes (diet Sea 1) and diet Sea 2 corresponds to Chalimus II and III. A detail of average caligus/tank can be reviewes in Table 4.

TABLE 4
Sampling of parasite loading (9 poat-infestation days)
Average Caligus: 24.3

| | | | | | | |
|---|---|---|---|---|---|---|
| Diet | Sea 1 | Sea 2 | Sea 1 | Sea 2 | Sea 1 | Sea 2 |
| Tank | 17 | 18 | 19 | 20 | 21 | 22 |
| Average Caligus | 26.0 | 25.8 | 25.1 | 22.3 | 24.4 | 22.6 |
| Standard Desv. | 10 | 9 | 8 | 7 | 9 | 7 |
| CV | 39.2 | 33.0 | 33.4 | 31.0 | 36.4 | 31.2 |
| Máx. | 50 | 46 | 42 | 37 | 50 | 36 |
| Mín. | 8 | 12 | 13 | 10 | 12 | 11 |
| Total Caligus | 1116 | 1134 | 1103 | 981 | 1073 | 993 |
| No./total | | | | | | |

A sampling of weight to final assay is performed to the totality of fishes/tank having an average weight per group of 442 g. The highest average weight was obtained to tank 21 (diet Sea 1) having 453.8 g and the lowest average weight was obtained to tank 18 (diet Sea 2) having 422.6 g. Details of this sampling is observed in Table 5.

TABLE 5
Sampling detail of final weight
Average weight: 442 g

| Diet | Sea 1 | Sea 2 | Sea 1 | Sea 2 | Sea 1 | Sea 2 |
|---|---|---|---|---|---|---|
| Tank | 17 | 18 | 19 | 20 | 21 | 22 |
| Average Weight (g) | 439.0 | 422.6 | 448.2 | 451.4 | 453.8 | 438.0 |
| Length (cm) | 32.9 | 32.7 | 33.2 | 33.1 | 33.1 | 33.1 |
| Standard Desviation | 97.9 | 74.7 | 79.4 | 81.1 | 87.9 | 72.6 |
| CV (%) | 22.3 | 17.7 | 17.7 | 18.0 | 19.4 | 16.6 |
| Máx. | 600 | 564 | 633 | 571 | 627 | 616 |
| Mín. | 250 | 238 | 302 | 233 | 240 | 254 |
| Fish No./tank | 43 | 44 | 44 | 44 | 44 | 44 |

A final assay sampling was performed (48 hrs after ended a second period of diet administration) and a final Caligus counting was considered to be performed to the totality of fishes/tank. The highest parasite loading was obtained to tank 19 (diet Sea 1) having 13.8 Caligus/fish while the lowest parasite loading was obtained to tank 20 (diet Sea 2) having 4.1 Caligus/fish. Details of the sampling can be observed in Table 6.

TABLE 6
final Caligus sampling
Average Caligus: 9.2

| Diet | Sea 1 | Sea 2 | Sea 1 | Sea 2 | Sea 1 | Sea 2 |
|---|---|---|---|---|---|---|
| Tank | 17 | 18 | 19 | 20 | 21 | 22 |
| Average Caligus | 12.1 | 7.6 | 13.8 | 4.1 | 12.6 | 5.3 |
| Standard Desv. | 5 | 4 | 5 | 3 | 7 | 2 |
| CV (%) | 43.2 | 56.1 | 34.5 | 66.6 | 53.1 | 44.8 |
| Máx. | 27 | 22 | 26 | 12 | 34 | 14 |
| Mín. | 2 | 1 | 3 | 0 | 4 | 2 |
| Caligus/tank | 484 | 309 | 575 | 171 | 535 | 216 |
| Detached by handling | 36 | 25 | 31 | 10 | 19 | 16 |
| Caligus Total/tank | 520 | 334 | 606 | 181 | 554 | 232 |

It observed, with respect to the parasite loading per treatment, that diet Sea 2 is obtained an average of 5.7 Caligus/fish compared to diet Sea 1 from which is obtained an average of 12.8 Caligus/fish. See FIG. 1.

Performed a parasite loading analysis as stage, it is observed that product causes certain effect in the life-cycle development (Table 7). To diet 1, 99% of the counted parasite in fish as average are in mobile adult stage (females, males and ovigerous females) was observed while to diet Sea 2 only 53% (average) was observed (counted parasite in fish found in mobile adult stage (females, males and ovigerous females) and 47% (average) remaining counted parasites delayed its development being in Chalimus III and IV stages. Also, parasites in Chalimus II (after 20 days the copopodite infestation) are even present in one of the replicas to diet Sea 2 (tank 18), was observed.

TABLE 7
Caligus number per developing Stage

| Diet | Sea 1 | | | Sea 2 | | |
|---|---|---|---|---|---|---|
| Tank | 17 | 19 | 21 | 18 | 20 | 22 |
| Chalimus I-II | 0 | 0 | 0 | 11 | 0 | 0 |
| Chalimus V | 4 | 2 | 1 | 144 | 84 | 109 |
| Mobile Adults | 516 | 604 | 553 | 168 | 97 | 123 |
| Parasite Total | 520 | 606 | 554 | 334 | 181 | 232 |
| % Mobile adults | 99.2 | 99.7 | 99.8 | 50.3 | 53.6 | 53.0 |
| % chalimus | 0.8 | 0.3 | 0.3 | 46.4 | 46.4 | 47.0 |

Performed parasite loading with respect to mobile adult group it is observed a post-treatment of the group presents certain differences between the administered experimental diet (Sea 2) and control (Sea 1) (FIG. 2). In control tanks having diet Sea 1, population is composed in a more homogeneous way between males and females (ovigerous females+females). Females (added as total) composing between 44 and 55% population (FIG. 5), proportion which is regularly present to control groups. As opposed, in tanks treated with dieta Sea 2, a major part of population is composed by male parasites and a very low ovigerous female loading is present. This shows that probably male individuals were resistant to the treatment and the most affected and sensitive population to the treatment were ovigerous females, which are present in a low percent (between 1.8 to 8.1% among its replicas, (FIG. 5).

In table 8 it is observed a summarize to recorded C. rogercresseyi counting and efficacy calculate, considering diet Sea 1 as control group. Achieved efficacy percent by 3 replicas of diet Sea 2 is 56.9%.

TABLE 8
Efficacy percent to experimental diet Sea 2

| | | | | Average | |
|---|---|---|---|---|---|
| | | Caligus | Average | Caligus/ | |
| | | Total/tank | Caligus/tank | treatment | |
| Treat- | | Post- | Post- | Post- | % treatment |
| ment | Tank | tratamiento | tratamiento | tratamiento | efficacy |
| Sea 2 | 18 | 334 | 7.6 | 5.7 | 56.9 ± 1.77 |
| | 20 | 181 | 4.1 | | |
| | 22 | 232 | 5.5 | | |
| Sea 1 | 17 | 529 | 12.1 | 12.8 | NA |
| (Con- | 19 | 606 | 13.8 | | |
| trol) | 21 | 554 | 12.6 | | |

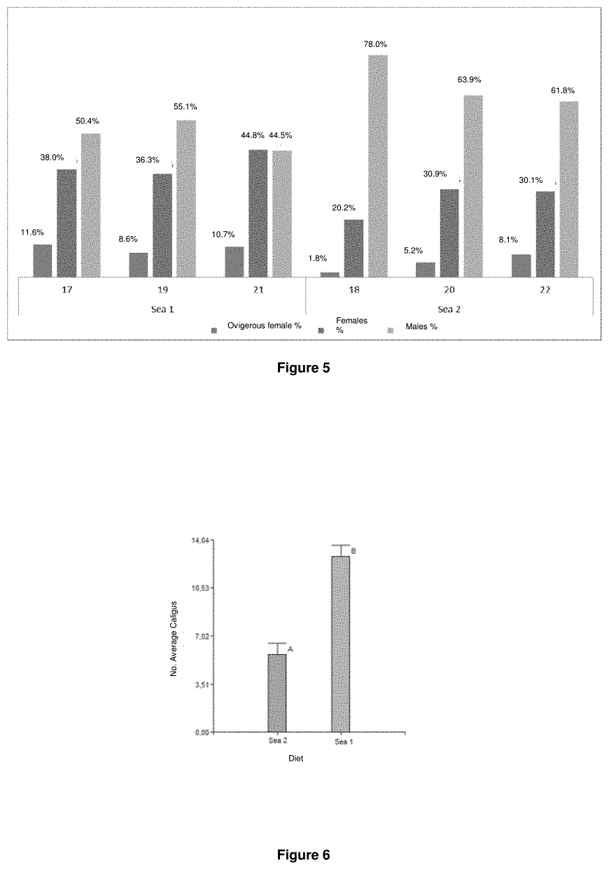

From the obtained data of parasite loading sampling, a variance statistical analysis was made verifying previously if data fulfil normality and homoscedasticity condition by Shapiro Wilks Test and Levene Test, respectively. Due to data fulfilled the normality and homoscedasticity condition, a one-way variance analysis was made applying comparative LSD Fisher Test, with which was analyzes if parasite loading medias (average caligus/fish) per treatment showed or not significant differences among experimental diets. To the analysis infostat statistical software was used (free version). Applied statistical analysis shows that parasite loading presents significant differences (p<0.05) between diet control (Sea 1) and evaluated experimental diet (Sea 2) (FIG. 6).

### Example 6: (In Vivo) Fish Administration/Feeding with the Additive of the Present Invention to Testing Safety and Efficacy in Caligus Control and Elimination

### Efficacy Assay of the Additive of the Present Invention

Ovigerous females were collected, and larvae spawned from its sacs. During 3 days culture were kept into a chamber under controlled environmental conditions (14° C., 8 mg/L O₂ and 32 psu salinity), up to obtaining infective copepodites. After 4 days, 20 fishes in tanks 1 and 16 were infected with Caligus rogercresseyi copepodites to be used in the efficacy test to the additive of the present invention in a doses of 2000 food ton. Parallelly, 20 fishes of tanks 18 and 19 were infected, keeping as control, only providing food. At day 7 infections were reviewed, and at day 8 feeding with food containing the additive of the present invention was started in doses of 2000 g/food ton, at satiety with ratio of 3% average body weight each tank. Evaluations of infections were performed at days 7 and 13 after started the feeding. Weight, length, condition index, fish mortality was evaluated at the beginning and end of assay. See Tables 9 y 10. Environmental variables (temperature and oxygen concentration) is daily evaluated. Consumed food having the additive and control food were daily evaluated by discounting the remaining food weight, from the one assigned as ration in each tank. Further, at days 7 and 13 after initiated the feeding, tissue samples for histology were collected, sacrificing 3 fishes in each efficacy tank, fixing with formaldehyde each sample. With this, Caligus stages present in such fishes were extracted to histological and microscopical analysis. Product efficacy was evaluated by two ways: a) obtaining mortality percent of parasites present at the ending of assay with respect to ones present at the beginning of the feeding with an additive.

% Efficacy=initial average−final average×100initial average

b) obtaining mortality percent of parasites present in fishes fed with an additive compared to fishes fed with food.

% Efficacy=average control−average additive×100 average control

Safety Assay of the Additive of the Present Invention

Parallelly to efficacy assay, safety of additive of the present invention is evaluated to be incorporated in food to Salmo salar in doses of 2000 g/food ton. To this, 20 fishes in two tanks (13 and 14) were used, being supplied with diet (food+additive) by 13 days and 20 fishes in a tank (15) as control (only fed with food without additive). Food ration was like the efficacy assay and, also the evaluation way of consumed food. Weight, length, condition index, fish mortality are evaluated at the beginning and end of assay. See Tables 9 and 10. Environmental variables (temperature and oxygen concentration) are daily evaluated. See Tables 9 and 10. At days 7 and 13 after started the feeding, tissue samples are collected for histology, sacrificing 3 fishes in each safety tanks and 6 fishes of control tank.

Assay was developed under similar environmental conditions to all the tanks with fluctuations not limiting the parasite or fish survival. Average thermal condition was 14° C., from day 4, date in which the feeding with the present additive was started. Oxygen concentration oscillated between 7 and 8.5 mg/L, with a dropped at the ending of assay in tanks having parasited fishes and fed with the present additive.

Physiological conditions of fishes used in the assay were similar at the beginning and end of assay, having a similar condition factor near to 1, specially parasited fishes and fed with the present additive. In Tables 9 and 10, weight, length, and condition factor to both samples (day 1, beginning, and day 22, ending) were summarized. Compared each variable between beginning and ending of assay, increase of fish weight and length are recorded to tanks wherein fishes were fed with the present additive and control food, remarking a greater increment in fished fed with the present additive (>15 g).

TABLE 9
Weight, length and condition factor of fishes in 7 assay tanks having the present additive

| | | Initial | | | | | | | | |
|---|---|---|---|---|---|---|---|---|---|---|
| | | Averg. | | | Standard Desv. | | | Var. Coec. | | |
| Treatment | Fish No. | Weight (g) | length (cm) | Fc | Weight (g) | Length (cm) | Fc | Weight (g) | Length (cm) | Fc |
| Safety Additive - | 20 | 55.3 | 17.9 | 1.0 | 5.7 | 0.5 | 0.1 | 10.3 | 3.0 | 7.5 |
| Replica 1 | | | | | | | | | | |
| Safety Additive - | 20 | 55.1 | 17.8 | 1.0 | 5.8 | 0.7 | 0.0 | 10.5 | 3.8 | 5.0 |
| Replica 2 | | | | | | | | | | |
| Control-Safety | 20 | 55.0 | 17.8 | 1.0 | 5.4 | 0.7 | 0.0 | 9.7 | 3.7 | 5.0 |
| Additive Diet | 20 | 55.8 | 17.9 | 1.0 | 6.5 | 0.7 | 0.1 | 11.7 | 3.7 | 6.1 |
| Efficacy 1 | | | | | | | | | | |
| Additive Diet | 20 | 55.5 | 17.9 | 1.0 | 5.3 | 0.5 | 0.1 | 9.6 | 2.9 | 5.6 |
| Efficacy 2 | | | | | | | | | | |
| Caligus Control | 20 | 43.7 | 16.7 | 0.9 | 2.3 | 0.4 | 0.1 | 5.2 | 2.5 | 5.6 |
| Efficacy 1 | | | | | | | | | | |
| Caligus Control | 20 | 44.2 | 16.7 | 1.0 | 2.6 | 0.4 | 0.1 | 5.8 | 2.2 | 5.7 |
| Efficacy 2 | | | | | | | | | | |
| | | Ending | | | | | | | | |
| Safety additive - | 17* | 66.0 | 19.2 | 0.9 | 7.0 | 0.5 | 0.1 | 10.6 | 2.5 | 6.2 |
| Replica 1 | | | | | | | | | | |
| Ssfety additive - | 17* | 71.7 | 19.4 | 1.0 | 7.7 | 0.7 | 0.0 | 10.7 | 3.7 | 3.8 |
| Replica 2 | | | | | | | | | | |
| Control-Safety | 14* | 63.9 | 19.1 | 0.9 | 8.3 | 1.1 | 0.1 | 13.1 | 6.0 | 8.0 |
| Additive Diet | 16** | 60.9 | 18.6 | 0.9 | 7.9 | 0.7 | 0.1 | 13.0 | 3.6 | 5.7 |
| Efficacy 1 | | | | | | | | | | |
| Additive Diet | 17* | 59.6 | 18.6 | 0.9 | 7.5 | 0.7 | 0.1 | 12.5 | 3.8 | 6.2 |
| Efficacy 2 | | | | | | | | | | |
| Control-Caligus | 0 | Mortality 5 days prior to the end of assay | | | | | | | | |
| Efficacy 1 | | | | | | | | | | |
| Control-Caligus | 17*** | 41.8 | 16.9 | 0.9 | 4.0 | 0.4 | 0.1 | 9.6 | 2.5 | 8.7 |
| Efficacy 2 | | | | | | | | | | |

*pending fishes sacrified by histology
**1 dead fisho + 3 histology
***dead by caligidosis

Safety Assay Results

Daily feeding was effective in all the tanks in which fishes were fed with the present additive. There was not mortality in fishes to two safety tanks neither in control tank where fishes fed with control diet, only 1 fish dead in one of the fed tanks having the present additive. Generally, sanitary condition of fishes without parasite (Safety Assay) and fed with the present additive was better to the recorded in fishes fed with control diet.

Efficay Assay Results

Periodically swimming and feeding fish activities were observed in different tanks, parasited fishes evidenced a more hyperactive swimming during infestations, to reduce it, lately. Together this, a daily feeding was effective in all the tanks in which fishes were fed with the presente additive, incluided parasited fishes. To compare infested fishes fed with the present additive and control fed, a better acceptace was obtained in fishes fed with the presen additive up to ended assay having a better general sanitary condition.

Salmo salar farmed by 13 days, fed with a diet including the present additive in a doses of 2000 grs per food ton, was effective, with growth in size and lenght.

Generally, sanitary condition of fishes having (Efficacy assay) parasites fed with the present additive, was better to the one recorded to parasited fishes and fed with control diet.

A daily fish food having additive and control food was supplied considering 3% body weight. In 4 efficacy assay tanks (2 tanks having parasited fishes+additive, 2 tanks having parasited fishes +control without additive), fishes were similarly fed, varying between 1 and 2%, decreasing the consumption subsequent days after sampling day 7, date in which fishes were extracted from tanks and kept under fast to be hanided.

Day 15 and day 22, sampling was performed, after days 7 and 13 fed with the present additive and control food.

In 4 tanks, 10 fishes per tank were sampled in a first sampling and a totality in a second sampling, which evidenced a high caligidosis, upper to 50 parasite per fish in the first one and at least 20 in the second one.

Fishes fed with the present additive showing a slightly lower parasitosis than control tanks (<10%) in the first sampling while at the ending of assay difference of 80% caligidosis were recorded to fishes fed with the present additive compared to controls.

In first sampling, predominant stages in fishes fed with the present additive were chalimus 3-4 (>85%), with some representative specimens at lowest developed stages (chalimus 1-2) and low number of adults (<15%). Predominant stages in fishes fed with control diet was chalimus 3-4 together adults, sharing similar percentages (40 to 50%) and an infimum lower developed stage (<5%).

At the end of the assay, predominant stages were adult in 4 tanks, including ovigerous females. However, the presence of chalimus in different stages was recorded in fishes fed with the present additive, which were not present in fishes of control tanks. Thus, the product affected the development in these stages, avoiding maturity into adults. See Table 9.

Mortality valuation of parasites within 13 days of feeding with the present additive was upper to 70%. Fishes fed with control food evidenced scarce mortality, recording at least 5% in a tank and null in the other one (Table 9).

Thus, together with the delay in maturity into adults in fishes fed with the present additive, a high mortality of chalimus stages was also observed, maturing into adults at least 30%. Calculating the efficacy of the present additive comparing the parasite abundance at the beginning and end and the ending of assay to each tank, values are near to 70%. While after compared the results of tanks with fishes fed having control additive, value was near to 80%.

TABLE 10
Average abundance of C. rogercresseyi stages in fishes fed with additive diet and control diet
in efficacy assay, together with the mortality percent within 13 days of experimentation.

| | | Fish | Day | | | | | | | | | | | | | | | | | | | % Mortality |
|---|---|---|---|---|---|---|---|---|---|---|---|---|---|---|---|---|---|---|---|---|---|---|
| Treatment | Tank | No | 1 | 2 | 3 | 4 | 5 | 6 | 7 | 8 | 9 | 10 | 11 | 12 | 13 | 14 | 15 | 16 | 17 | 18 | 19 | 13 days |
| Additive | 1 | 20 | 100 | | | 61 | | | | | | | | 50 | | | | | | | 17 | 72.1 |
| | 16 | 20 | 100 | | | 63 | | | | | | | | 57 | | | | | | | 16.5 | 73.8 |
| control | 18 | 20 | 100 | | | 89 | | | | 102 | | | | 75 | | | | | | | 86 | 3.4 |
| | 19 | 20 | 100 | | | 57 | | | | 62 | | | | 63 | | | | | | | 80 | 0.0 |

| Feeding | | Feeding | | | Feeding | |
|---|---|---|---|---|---|---|
| sampling | sam- | | sam- | | | |
| 5 fishes | pling | | pling | | | |
| sampling | | | | sam- | | |
| 10 fishes | | | | pling | | |
| sampling | | | | | | sam- |
| fish Total | | | | | | pling |

Days 1 to 3 = copepodites
Additive, days 4 to 11 = chalimus I-II. days 12 to 18 = chalimus III-IV. Day 19 = adults + HO
Control, days 4 to 7 = chalimus I-II. days 8 to 11 = chalimus III-IV. days 12 to 18 = chalimus III-IV + AM. Day 19 = adults + HO

During sampling made on days 7 and 13 of feeding with the present additive, samples in different parasite stages were collected, in fishes fed with diet containing the present additive and fishes fed only with food as control. Samples were preserved in fixing agents (formaldehyde, glutaraldehide and alcohol) to perform a subsequent microscopical and histological analysis.

A first analysis of samples included reviewing the gonad presence, body features and ovigerous sacs, with stereoscopic magnifying glasses and microscopes, in adults collected in the sampling day 22, ending of assay. Results of these reviewes were:
Sexual ratio of parasite adult was similar in fishes fed with the present additive and fishes fed with control, 60% females and 40% males;
Adult parasite of both genders, in fishes fed either with food containing the present additive or only food, presenting formed gonads and similar features (ovaries and oviducts in females, and testicles and spermatophores in males);
Parasite females of fishes fed with control were fertilized by males, recording 100% gravity (genital segment with fulfilled oviducts of vitellogenic oocytes) or with ovigerous sacs, remarking a wide genital. In fishes fed with additive, the most of the Caligus females did not evidence to be fertilized, in 80% genital segments were small, without oviducts having vitellogenic oocytes.
Comparing body chinitization of adult parasite, differences between gender was observed, recording a greater thickness in parasites of fishes fed with control with respect to parasites of fishes fed with the present additive, wherein the lowest chinitized by transparence let internal organs under evidence;
Ovigerous sacs in parasites of fishes fed with control presented more than 40 eggs per sac, whereas in caligus of fishes exposed to the present additive, a number was lower 10.

After 13 days of experimentation with the present additive at a dosis of 2000 grams per food ton, was observed that:
In the 1st days of feeding, fishes evidenced a high parasitosis, with differences in parasite dominant stages, between fished fed with the present additive and fishes fed with control food. A delay in development of juvenile stages (chalimus 1 to 4) is evidenced in parasites of fishes fed with food fish containing the present additive.
To conclude assay, at day 13 after started feeding, adult parasites to both genders are recorded and ovigerous females in fishes fed with diet containing the present additive and control. However, the efficacy of the present additive was demonstrated since in both tanks where fishes were fed with supplemented fed, i.e., with food containing the present additive, the same showed 20% parasite load/presence compared to fishes fed with control food.
Parasite life cycle is recorded in 15 days from copepodites to adults, to fishes either fed with diet containing the present additive or control food and 18 days of copepodites to ovigerous females. Thus, the additive of the present invention acted in the parasite metamorphosis—without adhering us to any theory, should be to the capability of inhibiting the chitin synthesis, limiting the development up to adults, without significantly affecting the cycle timing.
Parasite achieving maturity up to adults, only a scarce percent is transformed in larvae generators, only 10% was ovigerous and pregnant females, in fishes fed with diet containing the present additive with 40% parasite of fishes fed with control.
Further, without adhering us to any theory, it is estimated that due to the participation of chitin generator glands (cement gland) in reproduction (Asencio, 2015. Caligidosis en Chile. Aprendiendo Acuicultura Compendio, Tomo II. Salmonexpert 100 pag.) either males as females, there would be an effect in the presence of pregnant or ovigerous females. Females and males evidenced matured organs but for achieving eggs fecundation and formation the participation of chitin gland present in genital segment of both genders is required, and the present additive would influence this physiological procedure. To test histological analysis of gonads and genital segment of male and female collected in this assay, is required to determine if there was female fecundation (presence of sperm in female spermatheca) and gland activity of cement gland (lower size).
Diet with the present additive of 13 days, allowed the mitigation of caligidosis in fishes infested with Caligus under controlled conditions, improving the sanitary condition of fishes of assay. Fishes were exposed to a high parasitosis (>50 caligus per fish), and after supplying the additive, parasitosis was drastically reduced, with effects also in fecundity of adult parasites and with evidence to optical microscope of chinitization differences, developing female genital segments and ovigerous sacs reduced in egg number.

### Example 7: Comparison of the Present Additive with Emamectin Benzoate in Combating Parasites in In Vitro Assays

A bioassay was developed to evaluate the present additive under controlled environmental conditions (14° C.) with sea water (32 ppm, 8 mg/L oxygen concentration) in plate.

Bioassay was extended for 48 h to observe the effect of the present additive in the swimming behavior and survival of ovigerous Caligus females compared to emamenctin benzoate, widely applied to the treatment used to parasitosis control. Bioassay ended 2 hours after stated due to the presence of a greater percent of affected parasites, considering to this the swimming behavior, appendages and intestinal tract movements and response to stimulate as touch with tweezers or water movement, to be exposed to the present additive versus a very slow and erratic behavior observed in parasites exposed to emamectin benzoate. Main bioassays results are showed in Table 11 and are summarized below:
67% female parasites were affected by the present additive, 33% of the remaining females showed an erratic and slow swimming behavior.
25% female parasites exposed to emamectin benzoate showed scarce swimming or response to external stimulus. However, 75% living females slowly swam, having a slow response to stimulus and adhered to experimentation vessels.
Between 87 and 100% parasites survived in control with solvents (methanol and DMSO) used to prepare each product and in sea water. Under these conditions females actively swam and readily responded to stimulus as bubbles of the aeration system or manipulation with tweezers.

TABLE 11

| | Ovigerous | | | | Affected |
|---|---|---|---|---|---|
| | females | | | | and dead |
| Treatment | (Total) | Vivas | Affected | dead | Total (%) |
| Phytogenic Product | 15 | 5 | 4 | 6 | 67 |
| Emamectin | 15 | 11 | 1 | 3 | 27 |
| Methanol | 5 | 5 | 0 | 0 | 0 |
| DMSO | 5 | 5 | 0 | 0 | 0 |
| Sea water | 15 | 13 | 0 | 2 | 13 |

Results evidences a fast action of the present additive in ovigerous females causing behavior changes from the first 60 minutes. At the end of assay, all the parasite submerged in the settled additive were recorded without recording water swimming.

There was a slower response with emamectin benzoate compared to the present additive, recording parasite female swimming in water, adhesion to vessels and swimming response after touched with tweezers. Due to there was no effect in parasite females only exposed to solvents (methanol and DMSO) as used to each parasiticide (present additive and emamectin benzoate), slowness and mortality would be a result to both evaluated products.

### Example 8: Mortality Evaluation of the Additive in Plate (In Vitro)

The objective of this experiment was evaluating the effectiveness in controlling the Caligus rogercresseyi embryo development inside ovigerous sacs, under laboratory-controlled conditions (assay in vitro). Thus, a doses 2 mg/L of the present additive was used and an assay comprising its dilution in organic solvent dimethylsulfoxide (DMSO) and sea water was prepared as detailed in Table 12. Assay is based in the protocol established by Kumar y col. 2012 (Kumar, A., Raman, R. P., Kumar, K. et al. (2012) Antiparasitic efficacy of piperine against Argulus spp. on Carassius auratus (Linn. 1758): in vitro and in vivo study. Parasitol Res 111, 2071-2076). To assay, the present additive is transformed in a paste while including this mixture in sea water, a granular solution is obtained, which is precipitated at bottom of glass vessels. To keep an homogeneous mixture vessel was manually agitated with a glass rod or strongly agitated. Prior assaying, a mixture of the present additive was performed and was selected 0,5 L supernatant, using this last to apply to ovigerous female parasite in 3 Petri plates, 20 mL. 3 caligus ovigerous females under initial embryo stage of eggs were used in each plate. Thus, 9 females were used in plates with the present additive and 9 in control with sea water. Assay was prolongated for 4 days, evaluating the maturity development of ovigerous sacs each day. Assay results shows that the present additive has an effect on the ovigerous sac maturity of the treated females, wherein at the endo of the study, 83% females have immature sacs, 17% having a medium sac maturity, and matured ovigerous sacs are observed in females treated at day 4 (Table 12).

TABLE 12
Ovigerous Sac Development Stage

| | Inmature | | Medium Maturitya | | Mature | |
|---|---|---|---|---|---|---|
| Day | Phytogenic | Sea water | Phytogenic | Sea water | Phytogenic | Sea water |
| 0 | 100 | 100 | 0 | 0 | 0 | 0 |
| 1 | 100 | 22 | 0 | 78 | 0 | 0 |
| 2 | 83 | 0 | 17 | 33 | 0 | 67 |
| 3 | 83 | 0 | 17 | 0 | 0 | 100 |
| 4 | 83 | 0 | 17 | 0 | 0 | 100 |

What is claimed:

1. Food additive for fish food to controlling parasitosis comprising a mixture of a 1st botanical extract selected from a nimbus or Indian margosa extract and a second botanical extract selected from pepper and a taste masking agent selected from a seaweed extract.

2. The additive of claim 1 wherein such seaweed is Palmaria palmata.

3. Fish food for controlling parasitosis comprising the additive of claim 1 in a fish food at a dose ranging 200-4000 grams per food ton.

4. The fish food of claim 3 wherein such dose ranging 500-2000 grams per food ton.

5. The fish food of claim 4 wherein such dose ranging 200-500 grams per food ton.

6. Use of the additive of claim 1 in the prevention of caligidosis.

7. The fish food of claim 4 wherein such fish food is a fish food powder premix.

8. The additive of claim 1 wherein such additive is an oil-in-oil powder additive.

9. The additive of claim 1 wherein such additive is a capsule.

10. The additive of claim 1 wherein such additive is a solution in gel.

11. The additive of claim 1 additionally comprising additional palates.

12. The additive of claim 1 wherein the weight ratio between the first botanical extract: second botanical extract: seawater taste masking agent is ranging 70:10:20 to 50:5:45.

13. The additive of claim 13 wherein the weight ratio between the first botanical extract: second botanical extract: seawater taste masking agent is 65:5:30.

14. Use of the additive of claim 1 in the control of parasitosis limiting the larvae development ad sterilizing parasite adult stages.

15. A method for combating/eliminating Caligus infestations in fish farming centers and ocean concessions including sterilizing Caligus comprising orally o topically supplying the additive of claim 1 to such fishes.
